(19) United States
(12) Patent Application Publication
(10) Pub. No.: US 2026/0007469 A1
(43) Pub. Date: Jan. 8, 2026

(54) MEDICAL HANDPIECE

(71) Applicant: Dong Hyuk SONG, Seoul (KR)

(72) Inventor: Dong Hyuk SONG, Seoul (KR)

(21) Appl. No.: 19/124,159

(22) PCT Filed: Oct. 24, 2023
(86) PCT No.: PCT/KR2023/016565

§ 371 Date: Apr. 24, 2025

(30) Foreign Application Priority Data
Oct. 31, 2022 (KR) 20-2022-0002535

(51) Int. Cl. A61B 18/22; A61B 18/00; A61B 18/20; A61N 1/05; A61N 1/36

(52) U.S. Cl. CPC A61B 18/22; A61N 1/0502; A61N 1/36017; A61B 2018/0047; A61B 2018/2005; A61B 2018/202; A61B 2018/225

(57) ABSTRACT

The present invention presents a medical laser device. The medical laser device according to one embodiment of the present invention comprises: a body, which can be gripped and has an accommodation space therein; a needle part which is coupled to a front end portion of the body such that same can be replaced, and which is inserted into the skin of a patient so as to provide a high frequency current; and at least one weight adjustment part positioned inside the body. Here, the weight adjustment part includes at least one weight, and the body includes: the accommodation space in which the weight is accommodated; and a cover fixed to one side of the accommodation space such that same is detachable.

### TECHNICAL FIELD

The present invention relates to a medical handpiece.

### BACKGROUND ART

Lasers have been used in various fields such as industrial, medical, and military fields. In particular, medical laser devices have been used in ophthalmic clinics, dental clinics, surgical operations, dermatologic clinics, and the like. For example, the medical laser devices may be used to treat lesions such as skin diseases or vascular diseases that occur on the skin by emitting lasers to the lesions. In addition, the medical laser devices have been used to perform various types of procedures on living tissues in the skin beauty treatment field.

In general, since the medical laser devices require precise work, the medical laser devices are manufactured in the form of handpieces so that users may grip the devices with their hands to use the devices, and various types of needle tips may be mounted on an end portion of the medical laser device according to skin conditions and purposes so as to perform a treatment. For example, a skin procedure scheme of inserting a needle into a dermis layer of the skin to directly or indirectly transmit high-frequency waves to the dermis layer momentarily while alleviating damage to epidermis of the skin, such as pore, scar, acne, redness, and pigmentation treatments, has been used.

In this case, in a procedure where the needle is inserted deep, for example, in a procedure where the needle is inserted by a depth of 1.0 mm or more, when a handpiece is too light, a back push phenomenon may occur so that the needle may come out before reaching a desired depth, which makes it difficult to achieve a desired procedure result. In addition, since the users have to press the handpiece hard with strength of their wrists or shoulders to perform treatments in order to prevent the back push phenomenon, a procedure speed may slow down, and pain may be caused in the wrists or shoulders of the users. When a weight of the handpiece is increased in order to solve the above problems, although the back push phenomenon may not occur, the wrists or shoulders of the users may be strained by the weight of the handpiece.

### DISCLOSURE

### Technical Problem

One object of the present invention is to provide a medical handpiece capable of minimizing strain on a shoulder and a wrist of a user.

However, technical objects to be achieved by the present embodiment are not limited to the technical objects described above, and other technical objects may exist.

### Technical Solution

As a technical solution for achieving the technical objects described above, according to one embodiment of the present invention, a medical handpiece includes: a body configured to be grippable, and having an accommodation space therein; a needle part replaceably coupled to a front end portion of the body, and inserted into skin of a patient so as to provide a high-frequency current; and at least one weight adjustment part located inside the body. In this case, the weight adjustment part includes at least one weight, and the body includes: the accommodation space in which the weight is accommodated; and a cover part detachably fixed to one side of the accommodation space.

### Advantageous Effects

According to the solution of the present invention described above, positions and the number of weights can be adjusted according to a type of a procedure or a practitioner so as to adjust a weight and a center of gravity of a medical handpiece, so that strain on a shoulder and a wrist of the practitioner can be minimized.

In addition, the weight of the handpiece can be directly adjusted so as to be suitable for the practitioner, so that procedure efficiency of the practitioner can be improved.

### DESCRIPTION OF DRAWINGS

FIG. 1 is a perspective view showing a medical handpiece according to one embodiment of the present invention.

FIG. 2 is an exploded perspective view showing the medical handpiece according to one embodiment of the present invention.

FIG. 3 is a view showing a weight adjustment part according to a first embodiment of the present invention.

FIG. 4 is a view showing a weight adjustment part according to a second embodiment of the present invention.

FIG. 5 is a view showing a weight adjustment part according to a third embodiment of the present invention.

FIG. 6 is a view showing a weight adjustment part according to a fourth embodiment of the present invention.

### BEST MODE

Hereinafter, an embodiment of the present disclosure will be described in detail with reference to the accompanying drawings so as to be easily practiced by a person having ordinary skill in the art to which the present disclosure pertains. However, the present disclosure may be implemented in various different forms without being limited to the embodiment described herein. In addition, in order to clearly describe the present disclosure, in the drawings, parts that are not related to the description are omitted, and like parts are given like reference numerals throughout the present disclosure.

Throughout the present disclosure, when some part is described as being “connected” to another part, this includes not only a case where the part is “directly connected” to the other part, but also a case where the part is “electrically connected” to the other part with another device interposed therebetween.

Throughout the present disclosure, when some member is described as being located “on” another member, this includes not only a case where the member makes contact with the other member, but also a case where another member exists between the two members.

Throughout the present disclosure, when some part is described as “including” some component, unless explicitly described to the contrary, it means that another component may be further included but not excluded. The terms that express degrees, such as “about” and “substantially”, used throughout the present disclosure have meanings that are at a numerical value or close to the numerical value when unique manufacturing and material tolerances are presented in a stated meaning, and are used to prevent unconscientious infringers from unfairly exploiting the disclosure in which an exact or absolute value is stated in order to help understanding of the present disclosure. Throughout the present disclosure, “step (that) . . . ” or “step of . . . ” does not mean “step for . . . ”.

Hereinafter, exemplary embodiments of the present invention will be described in detail with reference to the accompanying drawings and the contents described below. However, the present invention is not limited to the embodiments described herein, and may be embodied in other forms. Throughout the present disclosure, like reference numerals refer to like elements.

The present invention relates to a medical handpiece 10, which may be a device for inserting a micro-needle 210 into the skin and providing high-frequency waves to apply local thermal damage into the skin so that microscopic wounds may be generated to promote skin regeneration.

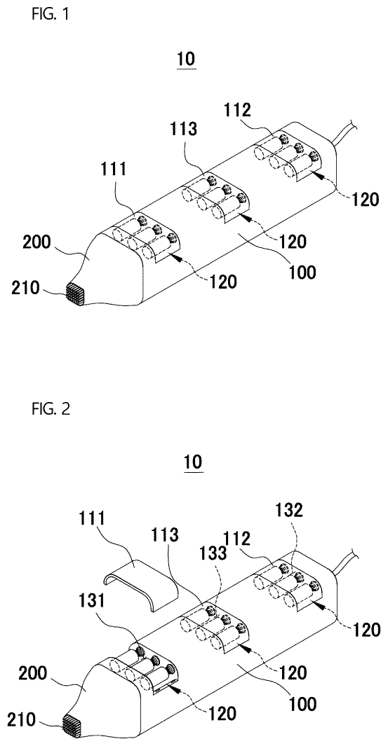

Hereinafter, the medical handpiece 10 (hereinafter referred to as a “handpiece 10”) according to one embodiment of the present invention will be described with reference to FIGS. 1 and 2.

The handpiece 10 may include a body 100, and a needle part 200 located at an end portion of the body 100.

The body 100 may be a component gripped by a practitioner, and may provide an installation space for other components. For example, the body 100 may have a columnar shape in which a sectional shape is a circular shape or a polygonal shape, but is not limited thereto.

In detail, the body 100 may be configured to be grippable, and the practitioner may insert the micro-needle 210 into the skin while moving forward toward the skin in a state of gripping the body 100. In addition, the micro-needle 210 may be removed from the skin while moving the body 100 rearward in a direction away from the skin.

The body 100 may have at least one installation hole (not shown) formed at a front end portion between both longitudinal end portions of the body 100 so as to provide an installation region for the needle part 200 that will be described below. For example, the needle part 200 may be replaceably coupled to the body 100, and the needle part 200 may be selectively replaced and used according to a type of a procedure.

The needle part 200 may be a component inserted into the skin to provide high-frequency waves to the skin.

In detail, while the needle part 200 is replaceably coupled to the body 100, the needle part 200 may move forward together with the body 100 during the forward movement of the body 100 by the practitioner so that the micro-needle 210 may be inserted into the skin of a procedure region. The micro-needle 210 may be heated by a heating member that is not shown in the drawings or provided with a high-frequency current by a high-frequency current member so as to treat the skin through thermal action.

The medical handpiece 10 may include a weight adjustment part 120 located inside the body 100 so as to adjust a center of gravity and a weight of the handpiece 10. In addition, the weight adjustment part 120 may include at least one weight (121, 122, 123), and each of the weights 121, 122, and 123 may have a cylindrical shape, a donut shape, and a spherical shape, which will be described in detail below.

The body 100 may include an accommodation space (131, 132, 133) in which the weight (121, 122, 123) is accommodated, and a cover part (111, 112, 113) detachably fixed to one side of the accommodation space (131, 132, 133). In addition, the accommodation space (131, 132, 133) and the cover part (111, 112, 113) may be located at at least one position selected from a front side, a center portion, and a rear side of the body 100. In other words, the accommodation spaces 131, 132, and 133 and the cover parts 111, 112, and 113 may be located at one position selected from the front side, the center portion, and the rear side of the body 100, may be located at two positions selected from the front side, the center portion, and the rear side of the body 100, or may be located at the front side, the center portion, and the rear side of the body 100, respectively. In detail, the body 100 may have a first accommodation space 131 formed at the front side of the body 100, in which a first cover part 111 covering the first accommodation space 131 is located, may have a second accommodation space 132 formed at the rear side of the body 100, in which a second cover part 112 covering the second accommodation space 132 is located, and may have a third accommodation space 133 formed at the center portion of the body 100, in which a third cover part 113 covering the third accommodation space 133 is located.

In addition, a plurality of accommodation spaces 131, 132, and 133 may be formed in parallel in a length direction as shown in FIG. 1, but are not limited thereto, and the accommodation spaces 131, 132, and 133 may be formed at various positions on a peripheral portion of the body 100. For example, the first accommodation space 131 may be located at a front upper portion of the body 100, the second accommodation space 132 may be located at a center lower portion of the body 100, and the third accommodation space 133 may be located at a rear upper portion of the body 100.

In addition, the cover parts 111, 112, and 113 may be completely separated from the body 100 as shown in FIG. 2, but are not limited thereto, and each of the cover parts 111, 112, and 113 may have one side that is rotatably coupled to the body 100 and an opposite side that may be fixed. In other words, in order to mount the weights 121, 122, and 123 of the weight adjustment part 120, the cover parts 111, 112, and 113 may be rotated or separated to open an inside of the body 100, and the weights 121, 122, and 123 may be inserted into the accommodation spaces 131, 132, and 133 so as to adjust a weight of the body 100.

In this case, the practitioner may adjust the weight of the handpiece 10 by adjusting the number of the weights 121, 122, and 123, and may adjust the center of gravity of the handpiece 10 by selectively mounting the weights 121, 122, and 123 on the front side and/or the rear side. Accordingly, the practitioner may adjust the number and mounting positions of the weights 121, 122, and 123 according to the type of the procedure and preference of the practitioner, so that strain on a shoulder and a wrist may be prevented, and the procedure may be performed more accurately.

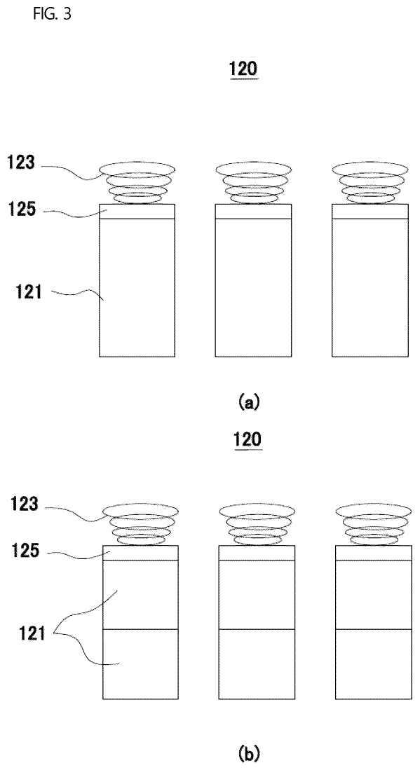

Hereinafter, weight adjustment parts 120 according to various embodiments of the present invention will be described with reference to FIGS. 3 to 6.

FIG. 3 is a view showing a weight adjustment part 120 according to a first embodiment of the present invention.

The weight adjustment part 120 may include at least one weight 121, and at least one elastic pressing part 123 for pressing the weight 121 with an elastic force. In addition, the elastic pressing part 123 may be located on one side of the accommodation space 131, and a plurality of elastic pressing parts 123 may be provided in a width direction. For example, the elastic pressing part 123 may be a coil spring, but is not limited thereto.

For example, as shown in FIG. 3(a), three elastic pressing parts 123 may be provided in the width direction, and the weight 121 may be selectively inserted into the three elastic pressing parts 123.

In addition, the weight adjustment part 120 may further include a support part 125 located at an end portion of the elastic pressing part 123 so as to press the weight 121. The support pat 125 may have the same shape as a sectional shape of the weight 121, and may make close contact with an end portion of the weight 121 so as to stably support the weight 121.

In addition, referring to FIG. 3(b), a plurality of weights 121 may be provided in a length direction. In other words, a plurality of weights 121 in which each of the weights 121 has a length that is shorter than a length of the weight 121 shown in FIG. 3(a) may be provided so that a weight may be adjusted more precisely.

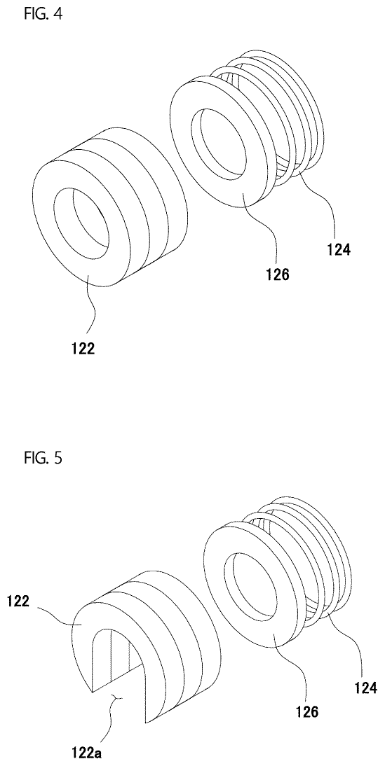

FIG. 4 is a view showing a weight adjustment part 120 according to a second embodiment of the present invention. As shown in FIG. 4, the weight 122 may be formed in a disc shape, and may have a donut shape formed by perforating a center portion of the weight 122. In this case, the practitioner may adjust a weight by stacking the weight 122. In addition, the support part 126 may have the same shape as the weight 122, but is not limited thereto. In addition, one elastic pressing part 124 may be provided on one side of the support part 126 so as to support the support part 126, but is not limited thereto, and a plurality of elastic pressing parts 124 may be located to elastically support the support part 126.

When the weight 122 has the donut shape, components mounted inside the body 100 may be located through a hole formed at a center of the weight 122, and a weight may not be biased to one side so that a center of gravity may be stably formed.

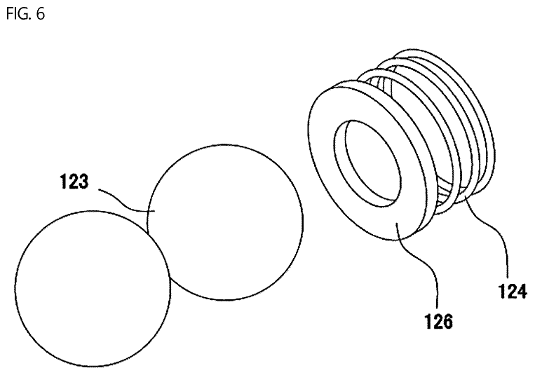

FIG. 5 is a view showing a weight adjustment part 120 according to a third embodiment of the present invention.

The weight 122 may have a donut shape, and may include an open part 122a formed by opening one side of the weight 122. In other words, when the weight 122 is mounted in the accommodation spaces 131, 132, and 133, components mounted in the inside may be inserted through the opened open part 122a and located at a center portion of the weight 122, so that the weight 121 may be easily installed. In addition, although it has been shown in FIG. 5 that a plurality of weights 121 are stacked, and open parts 122a are aligned with each other, the weights 121 may be mounted to allow directions of the open parts 122a to be different from each other so that a weight may not be biased.

FIG. 6 is a view showing a weight adjustment part 120 according to a fourth embodiment of the present invention.

The weight 123 may have a spherical shape. In addition, the support part 126 may have a donut shape, and may stably support the weight 123 having the spherical shape through a hole formed at a center of the support part 126. However, the configuration is not limited thereto, and when the weight 123 has the spherical shape, a groove having a hemispherical shape may be formed to allow the weight 122 having the spherical shape to be seated inside the accommodation spaces 131, 132, and 133, so that the weight 123 may be stably accommodated even without the elastic pressing part 124 and the support part 126.

Although each of the weights 121, 122, and 123 according to the present invention may have the cylindrical shape, the donut shape, the donut shape having the open part 122a as described above, the configuration is not limited thereto, and each of the weights 121, 122, and 123 may have various shapes such as a rectangular parallelepiped shape, a regular hexahedron shape, and an elliptical sphere shape. In addition, an inside of each of the accommodation spaces 131, 132, and 133 in which the weight 122 is accommodated may be formed to correspond to the shape of the weight 122 so that the weight 122 may not move inside the accommodation spaces 131, 132, and 133.

The above description of the present invention is for illustrative purposes, and it will be understood by a person having ordinary skill in the art to which the present invention pertains that the present invention can be easily modified in other specific forms without changing the technical idea or essential characteristics of the present invention. Therefore, the embodiments described above are illustrative in all aspects, and are not to be construed as limiting. For example, each component described in a single form may be implemented in a distributed manner, and similarly, components described as being distributed may be implemented in a combined form.

The scope of the present invention is defined by the claims described below rather than the detailed description, and the scope of the present invention is to be interpreted as encompassing all changes or modifications derived from the meaning and scope of the and equivalent concepts thereof.

### DESCRIPTION OF REFERENCE NUMERALS

10: Medical handpiece
100: Body
111, 112: Cover part
131, 132: Accommodation space
120: Weight adjustment part
121, 122: Weight 122a: Open part
123, 124: Elastic pressing part 125, 126: Support part
200: Needle part 210: Micro-needle

1. A medical handpiece comprising:
a body configured to be grippable, and having an accommodation space therein;
a needle part replaceably coupled to a front end portion of the body, and inserted into skin of a patient so as to provide a high-frequency current; and
at least one weight adjustment part located inside the body,
wherein the weight adjustment part includes at least one weight, and
the body includes:
the accommodation space in which the weight is accommodated; and
a cover part detachably fixed to one side of the accommodation space.

2. The medical handpiece of claim 1, wherein the accommodation space and the cover part are located at at least one position selected from a front side, a center portion, and a rear side of the body.

3. The medical handpiece of claim 1, wherein the weight adjustment part includes:
at least one elastic pressing part located on one side of the accommodation space so as to press the weight with an elastic force.

4. The medical handpiece of claim 3, wherein the weight adjustment part further includes:
a support part located at an end portion of the elastic pressing part so as to press the weight.

5. The medical handpiece of claim 1, wherein the weight has a cylindrical shape.

6. The medical handpiece of claim 1, wherein the weight has a donut shape.

7. The medical handpiece of claim 1, wherein the weight has a spherical shape.

8. The medical handpiece of claim 6, wherein the weight includes an open part formed by opening one side of the weight.
